(19) United States
(12) Patent Application Publication
(10) Pub. No.: US 2026/0290493 A1
(43) Pub. Date: Sep. 24, 2026

(54) MACHINE LEARNING ENABLED CONFORMATION-AWARE PROTEIN STRUCTURE PREDICTION

(71) Applicant: Genentech, Inc., South San Francisco, CA (US)

(72) Inventors: Franziska SEEGER, Seatlle, WA (US); Frédéric Alexandre DREYER, Brooklyn, NY (US)

(21) Appl. No.: 19/572,746

(22) Filed: Mar. 19, 2026

Related U.S. Application Data
(60) Provisional application No. 63/775,652, filed on Mar. 21, 2025.
(60) Provisional application No. 63/809,081, filed on May 20, 2025.
(60) Provisional application No. 63/840,407, filed on Jul. 8, 2025.
(60) Provisional application No. 63/977,665, filed on Feb. 6, 2026.

(51) Int. Cl. G16B 15/00; G06F 30/27; G06N 20/20; G16B 40/00

(52) U.S. Cl. CPC G16B 15/00; G06F 30/27; G06N 20/20; G16B 40/00

(57) ABSTRACT

A method for conformation aware protein structure prediction may include receiving an amino acid residue sequence of an input protein molecule, such as at least a portion of an antibody, a nanobody, or a T-cell receptor (TCR). A representation of the amino acid residue sequence of the input protein molecule may be determined to include a conformation token specifying a conformation state of the input protein molecule. The conformation state of the input protein molecule may be an unbound state or a bound state. A protein structure computation model may be applied to determine, based on the representation of the amino acid residue sequence of the input protein molecule, a conformation of the input protein molecule in the conformation state specified by the conformation token. Related systems and computer program products are also provided.

### CROSS REFERENCE TO RELATED APPLICATIONS

This application claims priority to U.S. Provisional Application No. 63/775,652; entitled “MACHINE LEARNING ENABLED PREDICTION OF ANTIBODY-SPECIFIC VARIABLE REGION STRUCTURES” and filed on Mar. 21, 2025, U.S. Provisional Application No. 63/809,081; entitled “MACHINE LEARNING ENABLED PREDICTION OF ANTIBODY-SPECIFIC VARIABLE REGION STRUCTURES” and filed on May 20, 2025, U.S. Provisional Application No. 63/840,407; “MACHINE LEARNING ENABLED PREDICTION OF ANTIBODY-SPECIFIC VARIABLE REGION STRUCTURES” and filed on Jul. 8, 2025, and U.S. Provisional Application No. 63/977,665, entitled “MACHINE LEARNING ENABLED PREDICTION OF ANTIBODY-SPECIFIC VARIABLE REGION STRUCTURES” and filed on Feb. 6, 2026, the disclosures of which are incorporated herein by reference in their entireties.

### TECHNICAL FIELD

The subject matter described herein relates generally to artificial intelligence and more specifically to machine learning based techniques for predicting the conformation of protein molecules.

### INTRODUCTION

A molecule is a group of two more atoms held together by chemical bonds. Molecules form the smallest identifiable unit into which a pure substance can be divided while still retaining the composition and chemical properties of that substance. Large molecules refer to those molecules that range between approximately 3000 Daltons and 150,000 Daltons in molecular weight. Large molecule therapeutics (also known as biopharmaceuticals, biotherapeutics, biologicals, or biologics) are often derivatives of natural human proteins, which modulate many essential cellular functions such as enzymatic reactions, transport of molecules, regulation and execution of a number of biological pathways, cell growth, proliferation, nutrient uptake, morphology, motility, intercellular communication, and/or the like. A single large molecule can include more than 1,300 amino acid residues linked by peptide bonds to form one or more polypeptide. Due to their size and complexity, large molecule therapeutics are recombinantly produced by engineered cells instead of being chemically synthesized like the majority of small molecule drugs. Moreover, large molecule therapeutics are usually delivered through injection or infusion due to the ineffectiveness of oral administration. The development of a large molecule therapeutics may entail designing one or more sequences of amino acid residues capable of binding to a target (e.g., a protein, a nucleic acid, and/or the like) with sufficient specificity and absent undesirable traits such as immunogenicity, self-association, instability, and/or the like.

### SUMMARY

Systems, methods, and articles of manufacture, including computer program products, are provided for machine learning based techniques for predicting the conformation (or three-dimensional structure of protein molecules or portions thereof. One or more properties of a protein molecule may be contingent on its conformation. For example, the binding affinity and specificity of an antibody towards an antigen (e.g., a viral antigen, a tumor antigen, and/or the like) may be contingent on the ability of the antibody to adopt a conformation that is complementary to that of the antigen. Accordingly, in some cases, a protein structure computation model may be trained to predict, based on the amino acid residue sequence of a protein molecule, the conformation (or three-dimensional structure) of at least a portion of the protein molecule, such as an antibody, a nanobody, a T-cell receptor (TCR), and/or the like. In some cases, the protein structure computation model may be trained to differentiate between the conformation of a protein molecule in an unbound state and that of the same protein molecule in a bound state. For instance, in some cases, the training dataset for the protein structure computation model may include the conformation of the same protein molecule in both the bound and the unbound state. As described in more details below, the protein structure computation model may be trained, based on the training dataset, to account for conformational flexibility of protein molecules, which are capable of undergoing substantial conformation changes when interacting or in complex with another molecule. Training the protein structure computation model for conformational awareness may result in the protein structure computation model exhibiting superior predictive accuracy and out-of-distribution generalizability compared to conventional protein structure computation models.

In one aspect, there is provided a system for machine learning enabled protein structure prediction. The system may include at least one data processor and at least one memory. The at least one memory may store instructions that cause operations when executed by the at least one data processor. The operations may include: receiving an amino acid residue sequence of an input protein molecule; determining a representation of the amino acid residue sequence of the input protein molecule, wherein the representation is determined to include a conformation token specifying a conformation state of the input protein molecule; and applying a protein structure computation model to determine, based at least on the representation of the amino acid residue sequence of the input protein molecule, a conformation of the input protein molecule in the conformation state specified by the conformation token.

In another aspect, there is provided a computer-implemented method for machine learning enabled protein structure prediction. The method may include: receiving an amino acid residue sequence of an input protein molecule; determining a representation of the amino acid residue sequence of the input protein molecule, wherein the representation is determined to include a conformation token specifying a conformation state of the input protein molecule; and applying a protein structure computation model to determine, based at least on the representation of the amino acid residue sequence of the input protein molecule, a conformation of the input protein molecule in the conformation state specified by the conformation token.

In another aspect, there is provided a computer program product for machine learning enabled protein structure prediction. The computer program product may include a non-transitory computer readable medium storing instructions. The instructions may cause operations when executed by the at least one data processor. The operations may include: receiving an amino acid residue sequence of an input protein molecule; determining a representation of the amino acid residue sequence of the input protein molecule, wherein the representation is determined to include a conformation token specifying a conformation state of the input protein molecule; and applying a protein structure computation model to determine, based at least on the representation of the amino acid residue sequence of the input protein molecule, a conformation of the input protein molecule in the conformation state specified by the conformation token.

In some variations, one or more features disclosed herein including the following features can optionally be included in any feasible combination.

In some variations, the conformation state of the input protein molecule is an unbound state comprising a three-dimensional structure adopted by the input protein molecule absent any interaction with another molecule.

In some variations, the conformation state of the input protein molecule is a bound state comprising a three-dimensional structure adopted by the input protein molecule interacting or in complex with another molecule.

In some variations, the representation of the amino acid residue sequence of the input protein molecule includes, for each constituent amino acid residue, a residue level encoding of the conformation token.

In some variations, the protein structure computation model includes an ensemble of structure module blocks operating on the representation of the amino acid residue sequence of the input protein molecule.

In some variations, each structure molecule block in the ensemble of structure module blocks updates, based at least on the representation of the amino acid residue sequence, one or more coordinates of one or more backbone atoms comprising each constituent amino acid residue in the input protein molecule.

In some variations, one or more coordinates of one or more sidechain atoms comprising each constituent amino acid residue are determined, using (i) idealized coordinates of a type of each constituent amino acid residue and (ii) one or more torsion angles the one or more sidechain atoms.

In some variations, the representation of the amino acid residue sequence of the input protein molecule further includes an embedding of the amino acid residue sequence generated by a protein language model (PLM).

In some variations, the representation of the amino acid residue sequence of the input protein molecule includes, for each constituent amino acid residue, a residue-level encoding of an antibody chain in which the amino acid residue is located.

In some variations, the representation of the amino acid residue sequence of the input protein molecule includes a positional encoding of a relative position of each constituent amino acid residue.

In some variations, the protein structure computation model includes an ensemble of independently trained machine learning models.

In some variations, each machine learning model in the ensemble of machine learning models is applied to determine the conformation of the input protein molecule.

In some variations, a plurality of outputs from the ensemble of machine learning models are aligned. A mean conformation is determined based on the aligned plurality of outputs from the ensemble of machine learning models. The conformation of the input protein molecule is determined to correspond to an output from a machine learning model in the ensemble of machine learning models that is closest to the mean conformation.

In some variations, the protein structure computation model includes a residual connection to every structure module block comprising the protein structure computation model to preserve the conformation token while the protein structure computation model operates on the representation of the input protein molecule.

In some variations, a training sample is generated to include an amino acid residue sequence of a sample protein molecule and a ground truth conformation of the sample protein molecule in an unbound state. An additional training sample is generated to include the amino acid residue sequence of the sample protein molecule and a ground truth conformation of the sample protein molecule in a bound state. A training dataset is generated to include the training sample and the additional training sample. The protein structure computation model is trained on the training dataset.

In some variations, one or more of the ground truth conformation of the sample protein molecule in the unbound state and the ground truth conformation of the sample protein molecule in the bound state comprise a crystallized protein structure.

In some variations, one or more of the ground truth conformation of the sample protein molecule in the unbound state and the ground truth conformation of the sample protein molecule in the bound state comprise a computationally generated protein structure.

In some variations, a protein design computation model is applied to generate the amino acid residue sequence of the input protein molecule. The protein design computation model generates the amino acid residue sequence of the input protein molecule by modifying (i) an amino acid residue sequence of a lead molecule having one or more known properties or (ii) a noise sequence without any known properties.

In some variations, the input protein molecule comprises at least a portion of an antibody, a nanobody, or a T-cell receptor.

In some variations, the input protein molecule comprises one or more of a complementarity determining region (CDR), a framework region, a variable region (or fragment antigen binding (Fab)), a constant region (or fragment crystallizable (Fc)), a light chain, or a heavy chain.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to antibodies and antibody variable regions (Fv), it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an example of a protein design system, in accordance with some example embodiments;

FIG. 2A depicts a flowchart illustrating an example of a process for machine learning enabled protein structure prediction, in accordance with some example embodiments;

FIG. 2B depicts a flowchart illustrating an example of a process for machine learning enabled protein structure prediction, in accordance with some example embodiments;

FIG. 3 depicts a block diagram illustrating the architecture of an example of a protein structure computation model, in accordance with some example embodiments;

FIG. 4 depicts a comparison of bound and unbound structure predictions and the corresponding bound and unbound ground truth structures, in accordance with some example embodiments;

FIG. 5 depicts a comparison of the conformational changes between bound and unbound antibody structures and the corresponding predicted conformational changes, in accordance with some example embodiments;

FIG. 6 depicts graphs illustrating the distribution of the number of structures by complementarity determining region heavy chain 3 (CDR-H3) edit distance to the closest matching datapoint, structural resolution, and CDR-H3 length, in accordance with some example embodiments;

FIG. 7 depicts a graph illustrating CDR-H3 root mean squared deviation (RMSD) as a function of edit distance to closest matching H3 loop in the benchmarking dataset, in accordance with some example embodiments;

FIG. 8 depicts graphs illustrating relative improvement in CDR-H3 RMSD from considering the best scoring prediction out of 10, 100, and 1000 seeds for Boltz-1 and Chai-1, in accordance with some example embodiments;

FIG. 9 depicts a graph illustrating the distribution of DockQ scores for predictions in the unbound and bound conformation docked in the ground truth antigen structure using Haddock3, compared against ABodyBuilder3, in accordance with some example embodiments;

FIG. 10 depicts graph illustrating the recall and precision of predicted hydrogen bond contacts for pairs of bound and unbound structures, in accordance with some example embodiments;

FIG. 11 depicts graphs comparing predicted CDR and framework RMSD for the protein structure computation model of the present disclosure and ABodyBuilder3 on the ImmuneBuilder test set of 34 antibodies, in accordance with some example embodiments;

FIG. 12 depicts graphs comparing predicted CDR and framework RMSD for the protein structure computation model of the present disclosure and NanoBodyBuilder2 on the ImmuneBuilder test set of 34 antibodies, in accordance with some example embodiments;

FIG. 13 depicts graphs illustrating predicted CDR and framework RMSD for the protein structure computation model of the present disclosure and TCRBuilder2+ on the ImmuneBuilder test set of 21 T-cell receptors (TCRs), in accordance with some example embodiments;

FIG. 14 depicts a graph comparing CDR-H3 RMSD as a function of edit distance to the closest matching H3 loop for Boltz-2, Boltz-2 without multi-sequence alignment (MSA) input, and the protein structure computation model of the present disclosure, in accordance with some example embodiments;

FIG. 15 depicts graphs comparing CDR-H3 RMSD as a function of edit distance to the closest matching H3 loop for different training dataset size and type of training data, in accordance with some example embodiments; and

FIG. 16 depicts a block diagram illustrating an example of a computing system, in accordance with some example embodiments.

When practical, similar reference numbers denote similar structures, features, or elements.

### DETAILED DESCRIPTION

Designing a protein sequence with one or more desired properties is a critical task in biomedicine and bioengineering. In the context of drug discovery, the protein sequence may be a protein therapeutic for treating, preventing, or curing diseases and medical conditions. Examples of protein therapeutics include antibodies, peptide hormones, growth factors, plasma proteins, enzymes, or hemolytic factors. The one or more desired properties may include binding affinity, binding specificity, functionality, and developability traits such as homogeneity, stability, solubility, and viscosity. The presence (and absence) of the one or more desired properties may determine the fitness of the protein sequence as a viable protein therapeutic. As such, the process of designing a protein sequence with the one or more desired properties typically includes two high-level phases: lead discovery (LD) followed by lead optimization (LO). The objective of lead discovery (LD) is to identify novel protein sequences exhibiting at least some level of fitness as a protein therapeutic. Those novel protein sequences then undergo lead optimization (LO), which aims to increase (or maximize) the fitness of the novel protein sequences identified through lead discovery (LD).

To accelerate drug development and reduce reliance on expensive wet lab resources, lead discovery (LD) as well as lead optimization (LO) may leverage computation tools. With three-dimensional structure playing a critical role in protein functionality, computational protein structure prediction may be especially beneficial to therapeutic protein design efforts. For example, in some cases, a protein structure computation model may be trained to predict, based on the amino acid residue sequence of a protein molecule (e.g., an antibody, a nanobody, a T-cell receptor (TCR), and/or the like), the three-dimensional structure adapted by the protein molecule. However, such protein structure computation models are generally required to operate in low data regimes where few crystallized protein structures are available for training. When trained with a small number of crystallized protein structures, conventional protein structure computation models tend to overfit (to those crystallized protein structures) and perform poorly when encountering protein structures that are underrepresented in the available training data. Moreover, despite the conformational flexibility of protein molecules, which can undergo substantial conformation changes when interacting or in complex with another molecule, conventional protein structure computation models are incapable of differentiating between the three-dimensional structures of protein molecules in bound and unbound states. Failure to account for conformational changes in protein structure prediction diminishes the predictive accuracy of conventional protein structure computation models, particularly for alternative protein conformations adopted, for example, by fold-switching proteins or through protein-protein interactions. In some cases, a protein molecule may be said to “adopt” a certain conformation (or three-dimensional structure) by at least adjusting or modifying, for example, through folding, its conformation or three-dimensional structure. In some cases, a protein molecule may be capable of adopting a multitude of different conformations due to its flexible nature. In some cases, the changes in the conformation of a protein molecule may be engendered by its interactions, including binding interactions, with other molecules in its proximate environment. For instance, instead of a precise conformation of a protein molecule in either the bound state or the unbound state, the output of conventional protein structure computation models tends to be a nonphysical average of the bound and unbound conformations of protein molecules or the most common conformational states.

The prediction of protein conformational changes engendered by interactions with another molecule (e.g., a ligand, another protein molecule, and/or the like) remains a central challenge in computational biology, directly impacting the ability to design and engineer effective therapeutics. Various embodiments of the present disclosure overcome the aforementioned limitations by at least training a protein structure computation model to differentiate between the three-dimensional structure of a protein molecule in an unbound state and that of the same protein molecule in a bound state (e.g., as a part of a protein-ligand complex, protein-protein complex, and/or the like). For example, in some cases, explicitly conditioning the protein structure computation model on conformation state may train the protein structure computation model to output distinct and accurate structural predictions for both the unbound and bound conformations from the amino acid residue sequence of at least a portion of the protein molecule. In some cases, the protein structure computation model may be capable of resolving the ambiguity that arises when training on structural databases in which multiple conformations are present for the same amino acid residue sequence. Previous approaches, which includes conventional protein structure computation models trained on undifferentiated data, risk predicting a nonphysical average or collapsing to the most common conformational state due to an ambiguous minimization objective. Through the introduction of a conformation token, various example embodiments of the protein structure computation model described herein is able to predict distinct state-specific minima in the energy landscape and can recapitulate known unbound/bound conformational changes. The practical implications of this ability includes improved property prediction for proteins exhibiting induced-fit recognition and more accurate docking through the selection of the correct starting point conformation. By providing an explicit conformation token, the protein structure computation model's structure module blocks learn to route information differently, attending to sequence-level features that correlate with different conformation states, which are otherwise averaged out in standard models. This phenomenon is supported by the distinct hydrogen bond networks predicted for the unbound and bound states, suggesting the protein structure computation model described herein may capture state-specific energy minima. Notably, various example embodiments of the protein structure computation model described herein is able to achieve high accuracy without requiring multiple sequence alignments (MSA). It should also be appreciated that the protein structure computation model described herein is also up to a hundred times faster at inference than existing general protein structure prediction models. These capabilities, combined with robust performance on out-of-distribution antibody sequences, represents a significant advance toward the in silico design and optimization of next-generation biologics.

In some example embodiments, the training dataset for the protein structure computation model may include the amino acid residue sequence of one or more sample protein molecules and the corresponding ground truth conformations in both a bound state and an unbound state. In this context, the unbound conformation of a protein molecule may refer to the three dimensional structure of the protein molecule in the absence of any interaction with another molecule. Contrastingly, the bound conformation of the protein molecule may refer to the three-dimensional structure adapted by the protein molecule when the protein molecule is interacting or in complex with another molecule. In some cases, training the protein structure computation model on both the bound and unbound conformations of the same protein molecule may enable the protein structure computation model to learn the distinctive structural motifs and folding patterns of different conformation states. Trained in this manner, the resulting protein structure computation model may provide a fast, accurate, and conformational-aware tool for protein structure prediction. Furthermore, the protein structure computation model may bridge the gap between static and dynamic views of protein-ligand and protein-protein interactions as well as demonstrate robust generalization to novel protein sequences. As such, various example embodiments of the protein structure computation model described herein may be deployed to accelerate the computational design, screening, and engineering of therapeutic proteins, including antibodies, nanobodies, T-cell receptors, and/or the like.

In some example embodiments, the ground truth conformations of the one or more sample protein molecules used to train the protein structure computation model may include crystallized protein structures determined through protein crystallization methodologies, such as X-ray diffraction/X-ray crystallography, cryogenic electron microscopy (CryoEM) (including electron crystallography and microcrystal electron diffraction (MicroED)), small-angle X-ray scattering, neutron diffraction, and/or the like. In some cases, the training dataset for the protein structure computation model may be augmented, for example, using computationally predicted conformations. For instance, in cases where the crystallized bound conformation of a sample protein molecule is available but not its crystallized unbound conformation, the unbound conformation of the sample protein molecule may be determined computationally to serve as the ground truth unbound conformation of the sample protein molecule.

In some example embodiments, the trained protein structure computation model may operate on a representation of a protein molecule to determine the conformation of the protein molecule in either a bound state or an unbound state. For example, in some cases, the protein structure computation model may operate on a representation of the protein molecule that includes, for each constituent amino acid residue, a conformation token that enables a disambiguation between bound and unbound conformations. In some cases, the conformation token may be provided as a label during the training of the protein structure computation model and as an additional input when the trained protein structure computation model is used for inference. During training, for example, the conformation token may explicitly labels protein structures as either bound or unbound, enabling the protein structure computation model to learn the distinct structural features of each conformation state. At inference, the conformation token may be specified to directly generate a prediction for the desired conformational state from an amino acid residue sequence. For instance, in some cases, to ensure this conformation token effectively propagates through the network, a residual connection may be added from the initial embedding to every structure module block in the protein structure computation model. In some cases, the representation of the protein molecule may include, in addition or instead of the conformation token specifying its conformation state, one or more additional tokens specifying other biological states, such as environmental pH, allosteric modulation, and/or the like. In some cases, these biological states may affect the conformation of the protein molecule. Accordingly, it should be appreciated that the inclusion of the corresponding tokens in the representation of the protein molecule may enable the protein structure computation model to differentiate between the conformation of the same protein molecule in different biological states.

In some example embodiments, in addition to the conformation token (or the residue-level encoding thereof), the representation of the protein molecule may include an embedding capturing, for example, the type (e.g., of the twenty canonical amino acid residues) and position (e.g., relative position) of each constituent amino acid residue. For instance, in some cases, the representation of the protein molecule may include an embedding of the amino acid residue sequence of the protein molecule generated by a protein language model trained to generate embeddings that reflect the underlying biology of protein molecules, such as the nexus between protein structure and function. In some cases, the representation of the protein molecule may include the pairwise distance between the constituent amino acid residues. In some cases, the representation of the protein molecule may include a positional encoding (e.g., one-hot encoding) of the relative positions of each amino acid residue in the amino acid residue sequence. In some cases, where the protein molecule is an antibody, the representation of the protein molecule may include an encoding (e.g., a residue-level encoding) of the chain (e.g., heavy chain, light chain) in which each amino acid residue in the protein molecule is located.

In some example embodiments, the protein structure computation model may operate on the representation of the protein molecule to determine the position of one or more atoms forming each constituent amino acid residue. For example, in some cases, the protein structure computation model may determine, based at least on the representation of the protein molecule, the coordinates (e.g., Cartesian coordinates (x, y, z)) of one or more backbone atoms in each constituent amino acid residue. In some cases, the position (e.g. Cartesian coordinates (x, y, z)) of one or more sidechain atoms may be determined using idealized coordinates and the torsion (or dihedral) angles (e.g., chi (χ) angles) between the sidechain atoms. As noted, in some cases, the representation of the protein molecule may include a residue-level conformation token differentiating between bound and unbound conformations. Furthermore, the protein structure computation model may be trained, using sample protein molecules that have been explicitly labeled as having an unbound or bound conformation, to implicitly learn the distinctive structural motifs and folding patterns that differentiate protein molecules based on the corresponding amino acid residue sequences. Accordingly, the protein structure computation model is able to predict the conformation of the protein molecule to a high degree of accuracy even if the protein molecule is outside of the distribution of the training dataset used to train the protein structure computation model.

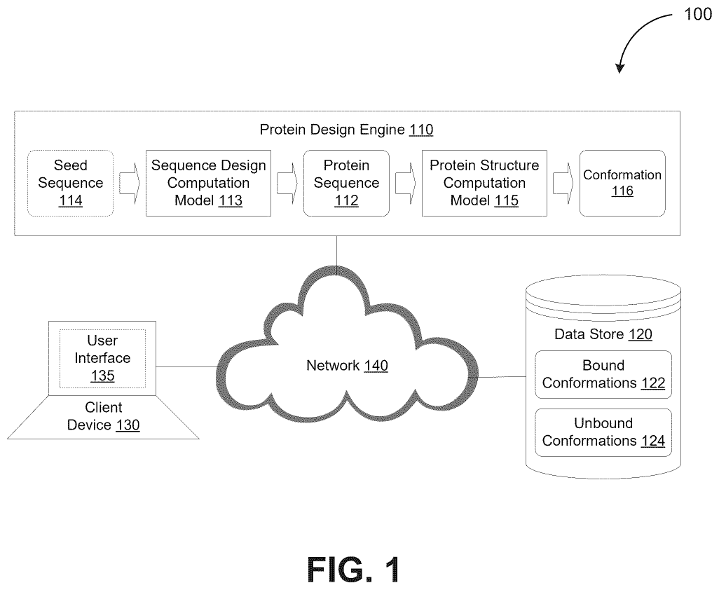

FIG. 1 depicts a system diagram illustrating an example of an antibody design system 100, in accordance with some example embodiments. Referring to FIG. 1, the antibody design system 100 may include a protein design engine 110, a data store 120, and a client device 130 including a user interface 135. In the example shown in FIG. 1, the protein design engine 110, the data store 120, and the client device 130 may be communicatively coupled via a network 140. In some cases, the data store 120 may be a database including, for example, a relational database, a NoSQL database, a columnar database, an objected-oriented database, a key-value database, a hierarchical database, a document database, a graph database, and/or the like. In some cases, the client device 130 may be a processor-based device including, for example, a workstation, a desktop computer, a laptop computer, a smartphone, a tablet computer, a wearable apparatus, and/or the like. In some cases, the network 140 may be a wired network and/or a wireless network including, for example, a local area network (LAN), a virtual local area network (VLAN), a wide area network (WAN), a public land mobile network (PLMN), the Internet, and/or the like.

Referring to FIG. 1, in some example embodiments, the protein design engine 110 may include a sequence design computation model 112 and a protein structure computation model 115. In some cases, the sequence design computation model 113 may be trained to generate one or more protein sequences, such as the protein sequence 112 shown in FIG. 1. In some cases, the protein sequence 112 may correspond to a protein molecule, such as an antibody, a nanobody, a T-cell receptor (TCR), and/or the like. In some cases, the protein sequence 112 may correspond to a portion of a protein molecule. For example, in some cases, the protein sequence 112 may correspond to one or more of a complementarity determining region (CDR), framework region, variable region (or fragment antigen binding (Fab)), constant region (or fragment crystallizable (Fc)), light chain, heavy chain, and/or the like.

Referring again to FIG. 1, in some cases, the sequence design computation model 113 may generate, based at least on a seed sequence 114, the protein sequence 112. For example, in some cases, the sequence design computation model 113 may be a machine learning model that has been trained to generate the protein sequence 112 by at least modifying (e.g., inserting, deleting, replacing, and/or the like) one or more amino acid residue in the seed sequence 114. In some cases, the seed sequence 114 may correspond to the amino acid residue sequence of a lead molecule, such as an antibody identified through an animal immunization campaign as having binding affinity towards a target antigen, in which case the sequence design computation model 113 may be applied to further optimize one or more properties (e.g., drug-like properties such as affinity, specificity, functionality, developability, and/or the like) by modifying the seed sequence 114. Alternatively, the sequence design computation model 113 may generate the protein sequence 112 de novo, meaning that the protein sequence 112 is generated “from scratch” rather than modifying an existing natural protein sequence, such as an antibody identified through an animal immunization campaign. In those instances, the sequence design computation model 113 may generate the protein sequence 112 without the seed sequence 114 or the seed sequence 114 may be a “noise” sequence, or a randomly ordered sequence of amino acid residues without any known properties.

In some example embodiments, the protein structure computation model 115 may be trained to determine, based at least on the protein sequence 112, the conformation 116 adapted by the protein sequence 112 in either a bound state or an unbound state. In this context, the conformation 116 of the protein sequence 112 in the unbound state may refer to the three-dimensional structure of the protein sequence 112 absent any interaction with another molecule. protein structure 116. Alternatively, the conformation 116 of the protein sequence 112 in the bound state may refer to the three-dimensional structure of the protein sequence 112 interacting or in complex with another molecule, such as a ligand, another protein molecule, and/or the like. As described in more detail below, in some cases, the protein structure computation model 115 may operate on a representation of the protein sequence 112 that includes a residue-level encoding of a conformation token specifying whether the protein structure computation model 115 is to determine the conformation 116 of the protein sequence 112 in a bound state or an unbound state. Moreover, in some cases, the protein structure computation model 115 may determine the conformation 116 of the protein sequence 112 by at least determining a position of one or more atoms in each constituent amino acid residue. For example, in some cases, the protein structure computation model 115 may determine the coordinates (e.g., Cartesian coordinates (x, y, z)) of one or more backbone atoms in each amino acid residue in the protein sequence 112 before the predicated torsion (or dihedral) angles (e.g., chi (x) angles)) between the sidechain atoms are used to determine the coordinates (e.g., Cartesian coordinates (x, y, z)) of the sidechain atoms from idealized coordinates.

In some example embodiments, the protein structure computation model 115 may include an ensemble of machine learning models (e.g., attention-based machine learning models). For example, in some cases, the protein structure computation model 115 may include an ensemble of multiple independently trained machine learning models, each of which trained using a different validation set selected from the available dataset. In some cases, the sample protein molecules included in each validation set may be selected provide a sufficiently large and representative set for monitoring convergence and preventing overfitting, while maximizing the amount of data available for training. In some cases, the outputs from the ensemble of machine learning models may be aligned. In some cases, a mean conformation may be determined based on the aligned conformations output by the ensemble of machine learning models. In some cases, the conformation 116 output by the protein structure computation model 115 may correspond to the output of the one machine learning model in the ensemble of machine learning models that is closest to the mean conformation.

In some example embodiments, the protein structure computation model 115 may operate on a representation of the protein sequence 112 that encodes the type (e.g., of the twenty canonical amino acid residues) and location of each constituent amino acid residue. In some cases, the representation of the protein sequence 112 may also include a residue-level encoding of a conformation token specifying that the protein structure computation model 115 is to determine the conformation 116 of the protein sequence 112 in an unbound state. In some cases, the representation of the protein sequence 112 may include an embedding of the constituent amino acid residues generated by a protein language model (PLM) trained to generate embeddings that reflect the underlying biology of protein molecules, such as the nexus between protein structure and function. In some cases, the representation of the protein molecule may include the pairwise distance between the amino acid residues in the protein sequence 112. In some cases, the representation of the protein molecule may include a positional encoding (e.g., one-hot encoding) of the relative positions of each amino acid residue in the protein sequence 112. In some cases, where the protein molecule is an antibody, the representation of the protein sequence 112 may further include an encoding (e.g., a residue-level encoding) of the chain (e.g., heavy chain, light chain) in which each amino acid residue in the protein sequence 112 is located.

In some example embodiments, the protein structure computation model 115 may be trained based on the three-dimensional structure of antibodies not bound to any antigens (e.g., unbound antibody structures 122 in the data store 120). Furthermore, in some cases, the structure computation model 115 may be trained based on the three-dimensional structure of antibodies bound to a target antigen (or bound antibody structures 124 in the data store 120). In some cases, each three-dimensional structure used to train the structure computation model 115 may include a label indicating whether the three-dimensional structure is that of the variable region (Fv) from an antibody not bound to any antigen (or an unbound structure) or an antibody bound to a target antigen (or a bound structure). In some cases, for antibodies where both the three-dimensional structure of the antibody not bound to any antigen and that of the same antibody bound to a target antigen are available, the structure computation model 115 may undergo contrastive learning such that the structure computation model 115 generates three-dimensional structures that are more similar to the three-dimensional structures of antibodies bound to a target antigen (or bound structures) and less similar to the three-dimensional structures of antibodies not bound to any antigens (or unbound structures).

In some example embodiments, the protein structure computation model 115 may be trained using a training dataset that includes the bound conformation 122 and the unbound conformation 124 of sample molecules that have been expressly labeled (or annotated) as such. For example, in some cases, each training sample in the training dataset may include the amino acid residue sequence of a sample protein molecule and the ground truth conformation of the sample protein molecule in at least one of a bound state and unbound state. In some cases, the training dataset for the protein structure computation model 115 may include one or more training samples including the amino acid residue sequence of a sample protein molecule and the ground truth conformations of the sample protein molecule in both a bound state and an unbound state. In some cases, training the protein structure computation model 115 on the bound and unbound conformation of the same protein molecules may lend conformational awareness to the protein structure computation model 115. For instance, unlike conformation-agnostic conventional protein structure computation models that output nonphysical averages or the most common conformational states, the conformation 116 of the protein sequence 116 generated by various example embodiments of the protein structure computation model 115 described herein may more precisely approximate the actual bound or unbound conformation of the protein sequence 112, even when the protein sequence 112 is an out-of-distribution input.

In some example embodiments, the ground truth conformations of the one or more sample protein molecules may include crystallized protein structures determined through protein crystallization methodologies, such as X-ray diffraction/X-ray crystallography, cryogenic electron microscopy (CryoEM) (including electron crystallography and microcrystal electron diffraction (MicroED)), small-angle X-ray scattering, neutron diffraction, and/or the like. However, in some cases, a crystallized protein structure may be available for only one of the bound and unbound conformations of some sample protein molecules. Accordingly, in some cases, the training dataset for the protein structure computation model may be augmented, for example, using computationally predicted conformations. For example, in cases where a crystallized protein structure is only available for one of the bound and unbound conformation of a sample protein molecule, a computationally predicted conformation may be used in its place.

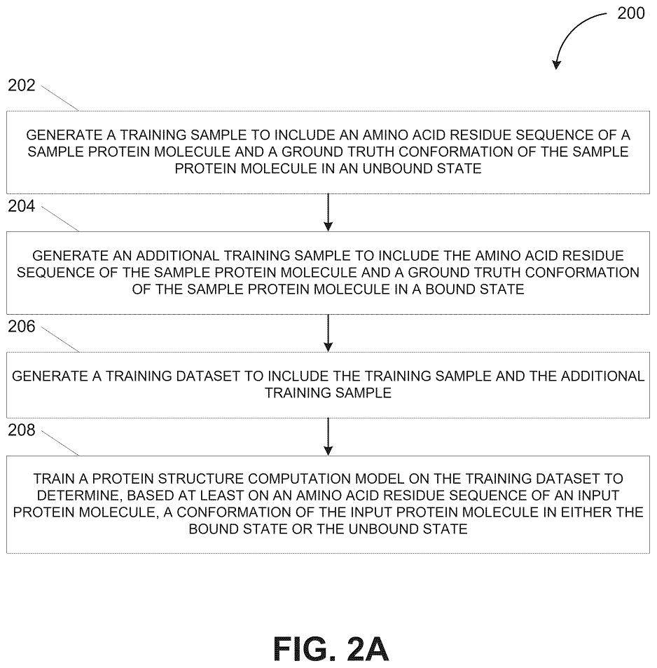

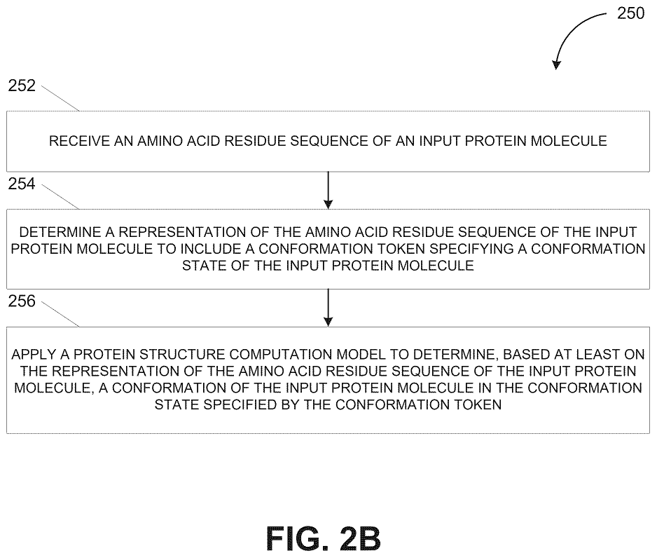

FIG. 2A depicts a flowchart illustrating an example of a process 200 for machine learning enabled protein structure prediction, in accordance with some example embodiments. Referring to FIGS. 1 and 2A, the process 200 may be performed by the protein design engine 110 to train, for example, the protein structure computation model 115. For example, in some cases, the protein structure computation model 115 may be trained to determine, based at least on the protein sequence 112, the conformation 116 adapted by the protein sequence 112 in either a bound or an unbound state. As described in more detail below, in some cases, explicitly labeled (or annotated) bound conformations 122 and unbound conformations 124 may be used to train the protein structure computation model 115 to learn the structural motifs and folding patterns associated with protein molecules in bound and unbound states. In some cases, the protein structure computation model 115 may be trained to exhibit conformational awareness, or the ability to differentiate between the bound and unbound conformation of the same protein molecule, such that the protein structure computation model 115 is able to determine a more precise approximation of the conformation 116 of the protein sequence 112 in either a bound or an unbound state.

At 202, a training sample is generated to include an amino acid residue sequence of a sample protein molecule and a ground truth conformation of the sample protein molecule in an unbound state. In some example embodiments, the training sample may be generated to include a representation of the amino acid residue sequence of the sample protein molecule. In some cases, the representation of the amino acid residue sequence of the sample protein molecule may encode the type (e.g., of the twenty canonical amino acid residues), location, and the conformational state of each constituent amino acid residue. For example, in some cases, the representation of the amino acid residue sequence of the sample protein molecule may include, for each constituent amino acid residue, a residue-level encoding of a conformation token specifying whether the protein structure computation model is to determine the conformation of the sample protein molecule in a bound state or an unbound state. In some cases, the representation of the amino acid residue sequence of the sample protein molecule may also include an embedding of the constituent amino acid residues generated, for example, by a protein language model (PLM) trained to generate embeddings that reflect the underlying biology of protein molecules (e.g., the nexus between protein structure and function). In some cases, the representation of the amino acid residue sequence of the sample protein molecule may include a positional encoding (e.g., one-hot encoding) of the relative positions of each amino acid residue in the sample protein molecule. In some cases, where the sample protein molecule is an antibody, the representation of the amino acid residue sequence of the sample protein molecule may further include, for each constituent amino acid residue, a residue-level encoding of the chain (e.g., heavy chain, light chain) in which the amino acid residue is located.

In some example embodiments, the training sample may be generated to include the ground truth conformation of the sample protein molecule in an unbound state absent any interaction with another molecule (e.g., a ligand, another protein molecule, and/or the like). In some cases, the ground truth conformation of the sample protein molecule may include, for each amino acid residue in the sample protein molecule, the position (e.g., Cartesian coordinates (x, y, z)) of one or more constituent atoms. For example, in some cases, the ground truth conformation of the sample protein molecule may include, for each amino acid residue in the sample protein molecule, the position (e.g., Cartesian coordinates (x, y, z)) of one or more backbone atoms, sidechain atoms, and/or the like. As described in more detail below, in some cases, while one training sample may be generated to include the ground truth unbound conformation of the sample protein molecule, an additional training sample may be generated to include the ground truth bound conformation of the same sample protein molecule.

At 204, an additional training sample is generated to include the amino acid residue sequence of the sample protein molecule and a ground truth conformation of the sample protein molecule in a bound state. In some example embodiments, an additional training sample may be generated to include a representation of the amino acid residue sequence of the same sample protein molecule and the ground truth conformation of the sample protein molecule in a bound state in which the sample protein molecule is interacting or in complex with another molecule (e.g., a ligand, a protein molecule, and/or the like). In some cases, the representation of the amino acid residue sequence of the sample protein molecule may include residue-level encodings of a conformation token indicating that the protein structure computation model is to determine the conformation of the sample protein molecule in a bound state. Moreover, the representation of the amino acid residue sequence may include an embedding encoding the type (e.g., of the twenty canonical amino acid residues) and location of each constituent amino acid residue including, in the case of antibodies, a residue-level encoding indicating the chain (e.g., heavy chain, light chain) in which each constituent amino acid residue is located.

At 206, a training dataset is generated to include the training sample and the additional training sample. In some example embodiments, the training dataset may be generated to include, for the same sample protein molecule, one training sample including the ground truth conformation of the sample protein molecule in a bound state and another training sample including the ground truth conformation of sample protein molecule in an unbound state. In some cases, one or both the ground truth bound conformation and the ground truth unbound conformation of the sample protein molecule may be a crystallized protein structure determined through, for example, protein crystallization methodologies, such as X-ray diffraction/X-ray crystallography, cryogenic electron microscopy (CryoEM) (including electron crystallography and microcrystal electron diffraction (MicroED)), small-angle X-ray scattering, neutron diffraction, and/or the like. In some cases, a crystallized protein structure may unavailable for one of the ground truth bound conformation and the ground truth unbound conformation of the sample protein molecule. In instances where a crystallized protein structure is unavailable for one of the ground truth bound conformation and the ground truth unbound conformation of the sample protein molecule, the absent ground truth conformation may be generated computationally. For example, in instances where a crystallized protein structure is available for the ground truth bound conformation of the sample protein molecule but not for the ground truth unbound conformation of the sample protein molecule, the ground truth unbound conformation of the sample protein molecule may be generated using computational methodologies, such as molecular dynamics simulations.

At 208, a protein structure computation model is trained on the training dataset to determine, based at least on an amino acid residue sequence of an input protein molecule, a conformation of the input protein molecule in either the bound state or the unbound state. In some example embodiments, the protein structure computation model may be trained to differentiate between the bound conformation and the unbound conformation of protein molecules using training samples that include both the ground truth bound conformation and the ground truth unbound conformation of one or more of the same sample protein molecules. In some cases, training the protein structure computation model on training samples with explicit labels (or annotations) for conformation state (e.g., bound or unbound) may enable the protein structure computation model to learn the distinctive structural motifs and folding patterns associated with different conformation states. In some cases, the resulting protein structure computation model may be conformational aware such that it is able to determine the precise bound or unbound conformation of the input protein molecule rather than, as in the case of conventional protein structure models, output a nonphysical average or the most common conformational state.

In some example embodiments, the protein structure computation model may be trained to determine, based at least on a representation of the amino acid residue sequence of the input protein molecule, the conformation of the input protein molecule in either the unbound state (e.g., without any interactions with another molecule) or the bound state (e.g., interacting or in complex with another molecule). In some cases, the representation of the input protein molecule may include a protein language model (PLM) embedding, a positional encoding, a residue-level chain encoding, and a residue-level conformation token encoding. In some cases, the protein structure computation model may be trained to determine, based at least on the representation of the input protein molecule, a position (e.g., Cartesian coordinates (x, y, z)) of one or more atoms (e.g., backbone atoms, sidechain atoms, and/or the like) in each constituent amino acid residue. For example, in some cases, the protein structure computation model may first determine the position (e.g., Cartesian coordinates (x, y, z)) of one or more backbone atoms in each constituent amino acid residue before the position (e.g., Cartesian coordinates (x, y, z)) of the corresponding sidechain atoms determined from idealized coordinates and the predicted torsion angles (or dihedral) angles (e.g., chi (χ) angles) between the sidechain atoms.

In some example embodiments, the training of the protein structure computation model may apply a curriculum learning strategy. For example, in some cases, application of the curriculum learning strategy may include first pretraining the protein structure computation model on a corpus of predicted protein structures and related experimental protein data. In some cases, the corpus for pretraining may include a broad corpus of non-antibody and/or non-TCR immunoglobulin-like domains. In some cases, the pretraining of the protein structure computation model may include knowledge distillation from predicted protein structures. In some cases, once the protein structure computation model is pretrained, application of the curriculum learning strategy may subsequently include training the protein structure computation model to gradually specialize towards more specific protein molecules, such as experimental antibody and T-cell receptor (TCR) data.

In some example embodiments, the training of the protein structure computation model may include three stages, at least some of which being associated with a different objective function. For example, in some cases, the first two stages of training may include reducing (or minimizing) a Frame Aligned Point Error (FAPE) loss along with a backbone torsion angle loss and Predicted Local Distance Difference Test (pLDDT) loss. In some cases, the Frame Aligned Point Error (FAPE) may be clamped at different thresholds (e.g., 10 Å and 30 Å) when computed between complementarity determining region (CDR) and framework region amino acid residues, or between loop and non-loop annotated amino acid residues in the case of immunoglobulin-like single domains. In some cases, the total loss term may be the sum of the average backbone Frame Aligned Point Error (FAPE) loss across each layer of the protein structure computation model, the full atom Frame Aligned Point Error (FAPE) loss from the final protein conformation predicted by the protein structure computation model, as well as side-chain and backbone torsion angle losses and a Predicted Local Distance Difference Test (pLDDT) loss. In some cases, the Predicted Local Distance Difference Test (pLDDT) loss may include cross-entropy loss on the discretized per-residue local Distance Difference Test based on Carbon-alpha atoms (IDDT-Cα).

In some cases, the third stage of training may include reducing (or minimizing) structural violation losses. For example, in some cases, structural violation losses may be reduced (or minimized) by at least penalizing bond length violations, bond angle violations, and steric clashes of non-bonded atoms. In some cases, the structural violation loss term may engender the generation of physically realistic protein structures. In some cases, the structural violation loss may supplement Frame Aligned Point Error (FAPE) loss at least because the latter alone does not guarantee correct local geometry. In some cases, the structural violation loss term may act as a finetuning operation, cleaning up unrealistic bond lengths and angles and resolving steric clashes introduced during the Frame Aligned Point Error (FAPE) loss driven training stages. In some cases, the hyperparameters for structural violation loss may include violation tolerances, clash radii, and/or the like.

In example embodiments, the protein structure computation model may be trained over multiple successive epochs. For example, in some cases, the protein structure computation model may undergo, for each of the three aforementioned stages of training, a certain number of epochs (e.g., 3000 epochs for the first stage, 2000 epochs for the second stage, and 1000 epochs for the third stage). In some cases, an epoch may include randomly sampling N sample protein molecules according to weighted probabilities assigned to each sample. In some cases, the value of N may be set in accordance to the total quantity of sample protein molecules available for training. In some cases, the probability assigned to each sample protein molecule may be inversely proportional to the cluster size to each sample protein molecule to improve out-of-distribution generalization. In some cases, matched pairs of bound and unbound conformations (of the same sample protein molecule) may be identified and sampled separately from those sample protein molecules for which only one conformation is available. In some cases, each batch of training samples may include a threshold quantity of matched pairs of bound and unbound conformations. In some cases, the training dataset may include the upsampling of certain protein molecules (e.g., nanobodies and T-cell receptors (TCRs) and matched pairs. In some cases, each stage of training may include a different proportion of sample protein molecules from various datasets.

FIG. 2B depicts a flowchart illustrating an example of a process 250 for machine learning enabled protein structure prediction, in accordance with some example embodiments. Referring to FIGS. 1 and 2B, the process 250 may be performed by the protein design engine 110 to apply, for example, the protein structure computation model 115 to determine the conformation 116 of the protein sequence 112. For example, in some cases, the protein structure computation model 115 may be trained to determine, based at least on the protein sequence 112, the conformation 116 adapted by the protein sequence 112 in either a bound or an unbound state. As noted, the protein structure computation model 115 may be trained on a training dataset with explicitly labeled (or annotated) bound conformations 122 and unbound conformations 124. In some cases, doing so may train the protein structure computation model 115 to learn the distinctive structural motifs and folding patterns associated with protein molecules in different conformation states, such as the unbound state in which a protein molecule is not interacting with another molecule and the bound state in which the protein molecule is interacting or in complex with another molecule. In some cases, the conformational awareness of the trained protein structure computation model 115, or its ability to differentiate between the bound and unbound conformation of the same protein molecule, may enable the protein structure computation model 115 to determine a more precise approximation of the conformation 116 of the protein sequence 112 in either a bound or an unbound state.

At 252, an amino acid residue sequence of an input protein molecule is received. In some example embodiments, the input protein molecule may include at least a portion of an antibody, a nanobody, a T-cell receptor (TCR), and/or the like. For example, in instances where the input protein molecule is an antibody, the input protein molecule may include one or more of one or more of a complementarity determining region (CDR), framework region, variable region (or fragment antigen binding (Fab)), constant region (or fragment crystallizable (Fc)), light chain, heavy chain, and/or the like. In some cases, the amino acid residue sequence of the input protein molecule may correspond to that of a lead molecule, such as an antibody identified through an animal immunization campaign as having binding affinity towards a target antigen. Alternatively, in some cases, the amino acid residue sequence of the input protein molecule may be generated computationally, for example, by a sequence design computation model modifying the amino acid residue sequence of a lead molecule or, in the case of de novo design, a noise sequence (e.g., a randomly ordered sequence of amino acid residues without any known properties).

At 254, a representation of the amino acid residue sequence of the input protein molecule is determined to include a conformation token specifying a conformation state of the input protein molecule. In some example embodiments, the representation of the amino acid residue sequence of the input protein molecule may include, for each constituent amino acid residue, a residue-level embedding of the conformation token specifying the conformation state of the input protein molecule. In some cases, the conformation token may provide, to the protein structure computation model applied to predict the conformation of the input protein molecule, an explicit indication to determine the conformation of the input protein molecule in either an unbound state or a bound state. In some cases, in addition to the residue-level embedding of the conformation token, the representation of the amino acid residue sequence of the input protein molecule may also include one or more of a protein language model (PLM) embedding, a positional encoding, and a residue-level chain encoding. For example, in some cases, the protein language model (PLM) embedding may be generated by a protein language model (PLM) trained to generate embeddings that reflect the underlying biology of protein molecules (e.g., the nexus between protein structure and function). In some cases, the positional encoding may encode the relative positions of the amino acid residues in the input protein molecule. In some cases, the representation of the amino acid residue sequence of the input protein molecule may include the pairwise distance between the amino acid residues in the input protein molecule. In some cases, where the protein molecule is an antibody, a residue-level chain encoding may be included with each amino acid residue to indicate, for the corresponding amino acid residue, the chain (e.g., heavy chain, light chain) in which the amino acid residue is located.

At 256, a protein structure computation model is applied to determine, based at least on the representation of the amino acid residue sequence of the input protein molecule, a conformation of the input protein molecule in the conformation state specified by the conformation token. In some example embodiments, the protein structure computation model may be a machine learning model, such as an attention-based machine learning models, or an ensemble of machine learning models, trained to determine, based at least on the representation of the amino acid residue sequence of the input protein molecule, the position of one or more atoms in each constituent amino acid residue. In some cases, the conformation token, which is provided as a label (or annotation) during the training of the protein structure computation model, may be used as an additional input at inference, to disambiguate between bound and unbound conformations. In some cases, the protein structure computation model may include a multilayer perceptron (e.g., a two-layer multilayer perceptron (MLP)), which operates on the protein language model (PLM) embedding of the input protein molecule first before it is concatenated with the remaining embeddings upon applying layer normalization. In some cases, pairwise edge features may be set to the one-hot encoding of relative positions of the amino acid residues in the input protein molecule concatenated with the residue-level chain embedding identifying the chain (e.g., light chain, heavy chain) in which each amino acid residue is located. In some cases, for each invariant point attention layer in the structure module in the protein structure computation model, this pairwise representation may be concatenated with a distance feature map and provided as input. It should be appreciated that in some cases, the core of the protein structure computation model may include multiple (e.g., 16 or a different quantity of) consecutive and independent structure module blocks, each of which operates to update the atomic coordinates of one or more amino acid residues and single representation through an invariant point attention layer. In some cases, the protein structure computation model may include a residual connection between the initial residue representation embedding and each structure module block to improve training convergence and ensures information from the conformation token is preserved throughout the network. In some cases, the representation of an amino acid residue from the last structure module block may be used to predict the coordinates (e.g., Cartesian coordinates (x, y, z)) of the backbone atoms in the amino acid residue. In some cases, the original input sequence may be used to reconstruct the coordinates (e.g., Cartesian coordinates (x, y, z)) of the sidechain atoms from idealized coordinates and using the predicted torsion (or dihedral) angles (e.g., chi (χ) angles) between the sidechain atoms. In some cases, uncertainties may be modeled through a Predicted Local Distance Difference Test (pLDDT) head, which predicts a projection of local confidence into a set of bins (e.g., a set of 50 bins).

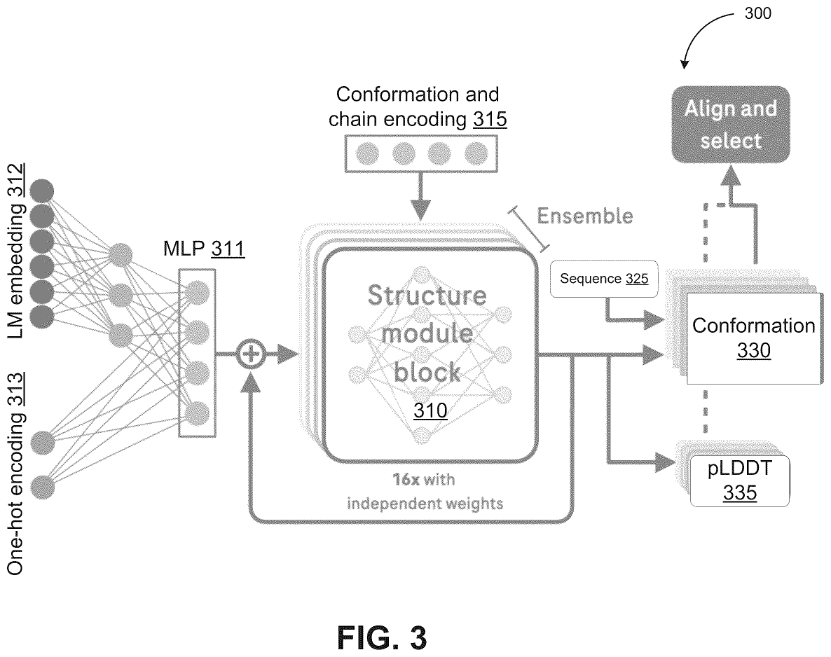

FIG. 3 depicts a block diagram illustrating the architecture of an example of a protein structure computation model 300, in accordance with some example embodiments. Referring to FIGS. 1 and 3, in some example embodiments, the protein structure computation model 300 may implement the protein structure computation model 115 shown in FIG. 1. Referring to FIG. 3, in some example embodiments, the protein structure computation model 300 may include an ensemble of multiple consecutive and independent structure module blocks 310. In the example shown in FIG. 3, the protein structure computation model 300 includes an ensemble of 16 structure module blocks 310 with independent weights. In some cases, the input of the protein structure computation model 300 includes a language model (PLM) embedding 312 (e.g., a protein language model (PLM) embedding), a one-hot encoding 313, and a conformation and chain encoding 315 of an amino acid residue sequence of an input protein molecule. In some cases, the conformation and chain encoding may include, for each amino acid residue in the input protein sequence, a residue-level enclosing of a conformation token distinguishing between bound and unbound protein structures. In some cases, the conformation token may be provided as a label (or annotation) during the training of the protein structure computation model 300 as well as used as an additional input at inference.

Referring again to FIG. 3, in some cases, the language model embedding 312 may be passed to a multilayer perceptron (MLP) 311 (e.g., a two-layer multilayer perceptron (MLP)) and concatenated with the other embeddings after applying a layer normalization. In some cases, pairwise edge features may be set to the one-hot encoding 313 of the relative positions of the amino acid residues in the input protein molecule and concatenated with the conformation and chain encoding 315, with the chain encoding indicating which chain (e.g., heavy chain, light chain) each amino acid residue belongs to. In some cases, for each invariant point attention layer in the structure module block 310, this pair representation may be concatenated with a distance feature map and provided as input. In some cases, the ensemble of structure module blocks 310 may update residue coordinates and single representation through an invariant point attention layer. In some cases, the protein structure computation model 300 may include a residual connection between the initial residue representation embedding and each structure module block 310, which improves training convergence and ensures information from the conformation token is preserved throughout the network. In some cases, the residue representation from the last structure module block 310 may be used to predict backbone atom coordinates (e.g., Cartesian coordinates (x, y, z)). In some cases, the original input sequence 325 may be used to reconstruct sidechain atoms from idealized coordinates using the predicted torsion (or dihedral) angles (e.g., chi (χ) angles) between the sidechain atoms. In some cases, uncertainties may be modeled through a Predicted Local Distance Difference Test (pLDDT) head 335 which, in the example shown in FIG. 3, predicts a projection of local confidence into 50 bins.

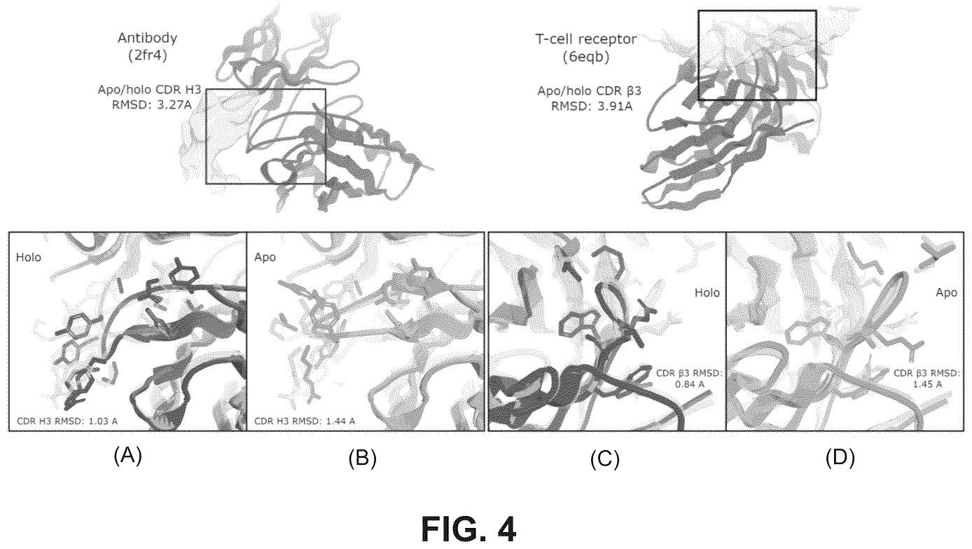

FIG. 4 depicts a comparison of bound and unbound structure predictions and the corresponding bound and unbound ground truth structures, in accordance with some example embodiments. As noted, various example embodiments of the protein structure computation model described herein may be capable of determining a precise conformation of a protein molecule in both an unbound state in which the protein molecule is not interacting with another molecule and in a bound state in which the protein molecule is interacting or in complex with another molecule. An example of this is shown in FIG. 4, with panels (A) and (B) showing the bound and unbound conformations of an antibody (PDB code: 2fr4) determined by the protein structure computation model superimposed on the corresponding ground truth conformations. Panels (C) and (D) depict the bound and unbound conformations of a T-cell receptor (PDB code: 6eqb) predicted by the protein structure computation model superimposed on the corresponding ground truth conformations.

### Experimental Examples

The performance of various example embodiments of the protein structure computation model described herein was evaluated by first assessing its capability to model the conformational transition between bound and unbound states. The accuracy of the structural predictions made by the protein structure computation model was then benchmarked against state-of-the-art models on a public test set before its generalization performance was tested on a large, private dataset of high-resolution antibody structures.

As noted, various example embodiments of the protein structure computation model described herein may be trained to recapitulate distinct conformational states of protein molecules. For example, from a single amino acid residue sequence, the protein structure computation model can predict both the bound and unbound conformation adapted by the amino acid residue sequence. This ability was validated by analyzing the conformational changes between 562 experimentally determined bound/unbound antibody pairs and comparing them to the changes predicted by the protein structure computation model described herein.

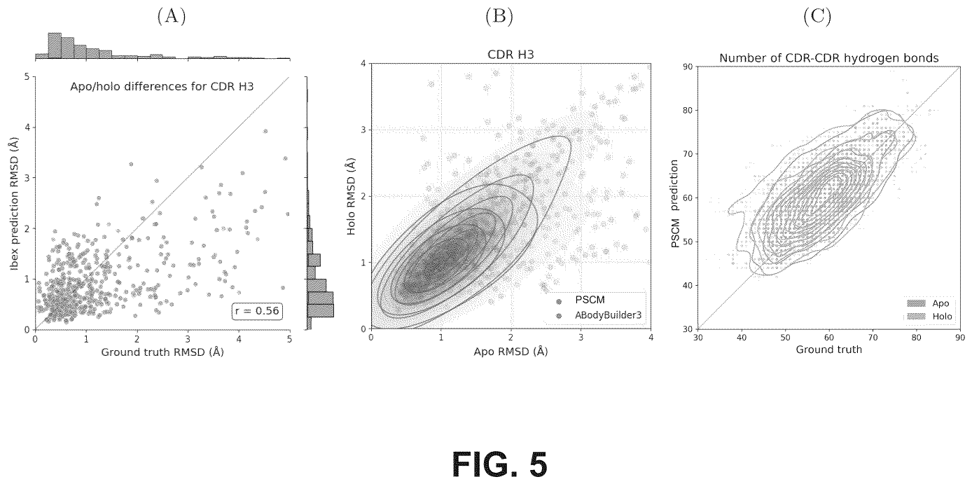

FIG. 5 depicts a comparison of the conformational changes between bound and unbound antibody structures and the corresponding predicted conformational changes, in accordance with some example embodiments. Panel (A) of FIG. 5 depicts the root mean square deviation (RMSD) in angstroms of the CDR H3 loop between bound and unbound structures, comparing the predictions of the protein structure computation model to the ground truth. Here the loop RMSD was computed by averaging over backbone atoms after aligning the heavy chain according to framework residues. Each point corresponds to a matching pair of experimentally determined structures with both conformations for the same variable domain sequence. The results in Panel (A) show that the protein structure computation model has a reasonable correlation with experimentally observed differences, and produces a similar distribution of overall conformational change. It is important to note here that 490 of the 505 known paired bound/unbound structures were included in the training, with the 15 test set structures annotated in light gray.

Panel (B) depicts the results of evaluating the performance of the protein structure computation model at recapitulating the respective ground truth structure. In Panel (B), the CDR H3 RMSD of the predictions made by the protein structure computation model is shown across all matching bound/unbound antibody pairs. The x coordinate gives the RMSD between predicted and experimental unbound structures, while the y coordinate gives the corresponding bound structure RMSD. In Panel (B), the performance of the protein structure computation model is compared against that of ABodyBuilder3. While each datapoint for the protein structure computation model corresponds to two distinct predictions, ABodyBuilder3 can only provide a single conformation for a specified sequence, and thus the same prediction was used to evaluate the RMSD to both the bound and unbound ground truth in that case. The contours of the kernel density estimation of the joint probability distribution function is shown in Panel (B), with the mode of the distribution indicated by a cross. As shown, the protein structure computation model achieves markedly better performance, with a narrower distribution that has fewer large RMSD outliers.

Finally, whether the predictions of the protein structure computation model reflect the corresponding biophysical states associated with conformational changes was assessed by quantifying the recapitulation of hydrogen-bond networks within the CDRs using the Rosetta ScoreFunction. Panel (C) in FIG. 5 shows that the protein structure computation model adequately recapitulates the expected number of hydrogen bonds in both the bound and unbound conformations, with a slight bias toward overproducing hydrogen bonds in the bound state between the number of hydrogen bonds observed in the ground truth structure compared to corresponding protein structure computation model predictions. Here, the predictions were evaluated on the same set of 562 matching bound/unbound pairs. As described in more detail below, precision and recall analysis of the predicted inter-CDR hydrogen bond contacts confirms the fidelity of these reproduced networks, indicating that the protein structure computation model correctly captures both the magnitude and connectivity of the intramolecular interactions stabilizing each conformation. The predicted local-distance difference test (pLDDT) of the protein structure computation model was also evaluated for the paired predictions. It is observed that framework regions consistently receive high pLDDT scores (>90) for both unbound and bound structural predictions, while CDR loops show greater variability. The pLDDT scores for the CDR H3 loop itself did not show a strong systematic difference between correct unbound and bound structural predictions.

To assess its performance against existing methods, the protein structure computation model was benchmarked on a standardized test set derived from the ImmuneBuilder suite, which includes antibodies, nanobodies, and TCRs. For consistent benchmarking, the respective test sets were combined, with assurance that no structures from a cluster containing a test set member were used during training. The performance of the protein structure computation model was compared against ESMFold, Chai-1, and Boltz-1, which are general protein structure prediction models, as well as against ABodyBuilder3, NanoBodyBuilder2, and TCRBuilder2+, three specialized models for antibodies, nanobodies and TCRs respectively. For Chai-1 and Boltz-1, a single seed and diffusion trajectory is used in all comparisons. Boltz-241 was excluded in this benchmark, as it uses a later PDB cutoff date for the curation of its training data, and thus was trained on most of the ImmuneBuilder test set. Furthermore, to mitigate data leakage, six structures from the nanobody test set for which a matching CDR H3 loop sequence was identified in the NanoBodyBuilder2 training or validation splits were removed. The RMSD is evaluated separately for each region by aligning each chain independently based on their framework residues and averaging over corresponding backbone atom RMSD.

As summarized in Table 1, the protein structure computation model demonstrates performance competitive with or exceeding other models, achieving the lowest mean RMSD for the TCR CDR β 3 (1.84 Å) and CDR α 3 (1.93 Å) loops. The protein structure computation model also has the second lowest RMSD for both antibody and nanobody CDR H3 loops. Chai-1 achieves marginally better results on the CDR H3 of antibodies, but poorly models the antibody CDR L3 loop as well as nanobodies and TCRs. Boltz-1, meanwhile, achieves the best results on the CDR H3 loop of nanobodies, but performs worse on the antibody CDR H3 loop and the TCR CDR β 3 loop. The protein structure computation model largely surpasses the performance of specialized models across all modalities. More detailed comparisons provided below.

TABLE 1

| | | CDR H1 | CDR H2 | CDR H3 | Fw H | CDR L1 | CDR L2 | CDR L3 | Fw L |
|---|---|---|---|---|---|---|---|---|---|
| Anti- | [symbol] | [symbol] | [symbol] | [symbol] | [symbol] | [symbol] | [symbol] | [symbol] | [symbol] |
| bodies | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] |
| Nano- | [symbol] | [symbol] | [symbol] | [symbol] | [symbol] | — | — | — | — |
| bodies | [symbol] | [symbol] | [symbol] | [symbol] | [symbol] | — | — | — | — |
| | [symbol] | [symbol] | [symbol] | [symbol] | [symbol] | — | — | — | — |
| | [symbol] | [symbol] | [symbol] | [symbol] | [symbol] | — | — | — | — |
| | [symbol] | [symbol] | [symbol] | [symbol] | [symbol] | — | — | — | — |
| | [symbol] | [symbol] | [symbol] | [symbol] | [symbol] | [symbol] | [symbol] | [symbol] | [symbol] |
| TCR [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] |

[symbol]

indicates data missing or illegible when filed

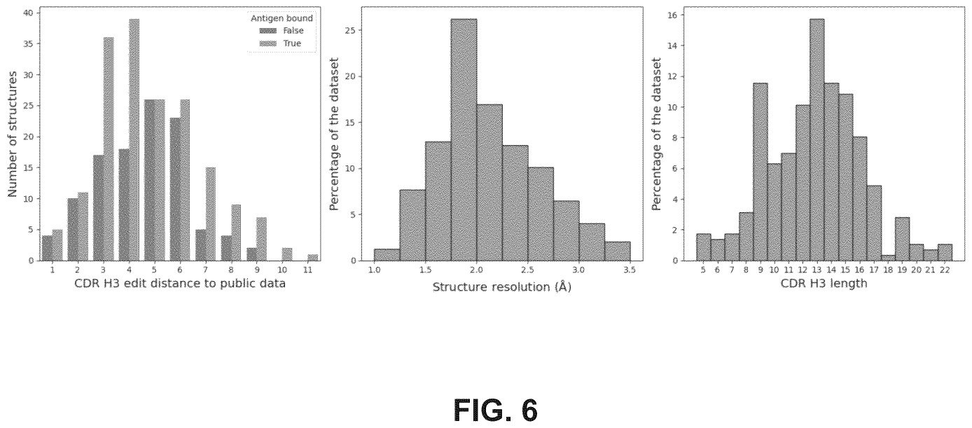

Various example embodiments of the protein structure computation model described herein was also evaluated on a curated dataset of 286 unique and novel antibody structures. Each structure in this dataset has a unique CDR H3 loop which differs by at least one amino acid from any known public antibody structure, and 45 have an edit distance of 7 or more from any CDR H3 loop in SAbDab. The length of the CDR H3 loops of these structures ranges from 5 to 22, with an average experimental resolution of 2.1 Å. Of those antibodies, 177 are bound to an antigen and 109 are in the unbound conformation. The distribution of number of structures by CDR H3 edit distance to the closest matching public datapoint, as well as the resolution and CDR H3 loop length distribution, are shown in FIG. 6.

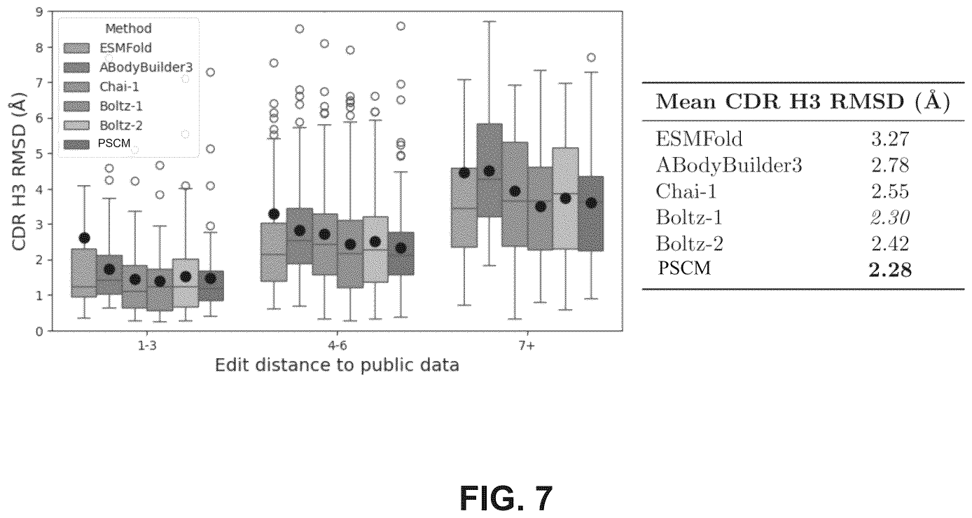

FIG. 7 shows the CDR H3 RMSD on this dataset as a function of edit distance to the closest matching CDR H3 loop in SAbDab. As shown, while ABodyBuilder3 achieves competitive performance on the test set, it is not as robust out-of-distribution (mean RMSD 2.78 Å) compared to more recent general protein structure prediction models like Boltz-1 (2.30 Å). In contrast, the protein structure computation model described herein shows comparable performance to Boltz-1, which may be attributed to distillation from predicted structures and the use of a broader corpus of training data. It has recently been shown that including antigen context can sometimes improve CDR H3 modeling accuracy in co-folding models. It is also interesting to note that ESMFold achieves comparable median performance to more recent models in the high edit distance region, indicating that there has been limited progress in out-of-distribution robustness of CDR loop modeling. However, ESMFold sometimes misfolds the variable region entirely, leading to high RMSD outliers and a larger average RMSD value compared to other methods. A comparison of inference compute time is provided below.

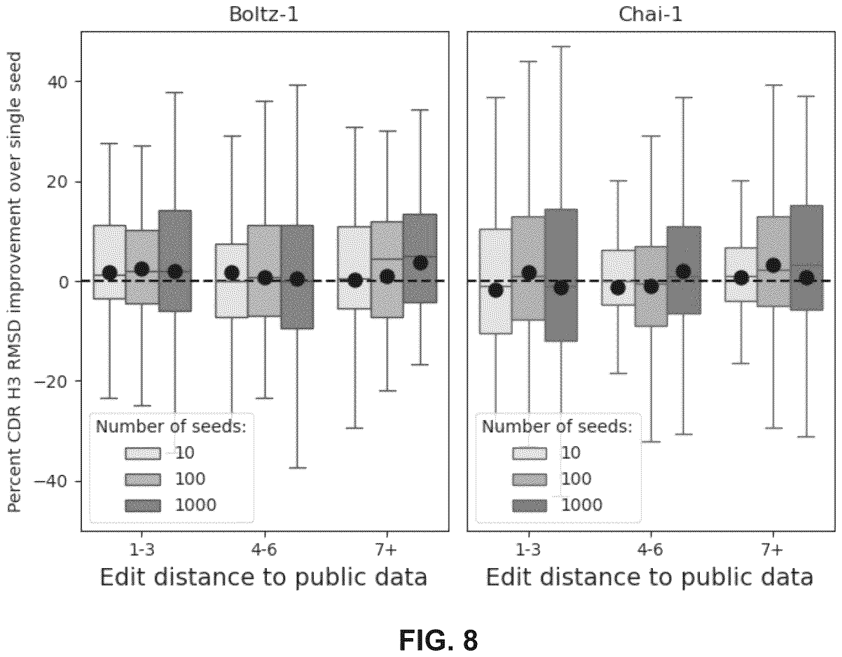

Due to their stochastic nature, diffusion-based structure prediction models such as AlphaFold3 can be sampled repeatedly, ranking results with their own confidence head. Notably, recent studies have shown that sufficient sampling can dramatically improve quality of antibody-antigen docking poses generated by the model. Whether similar scaling behavior could be observed in the modeling accuracy of the CDR H3 loop was investigated. In FIG. 8, Boltz-1 and Chai-1 was evaluated on a private dataset, sampling up to 1000 structures for each sequence, using distinct seeds and a single diffusion trajectory per seed. For each sequence, the CDR H3 RMSD between the highest confidence prediction and a single seed prediction was evaluated. In contrast to previous docking experiments, very little improvement in prediction accuracy was observed when scaling to 1000 seeds. Both models also mostly do not predict varying conformations of the loop backbone for the same sequence, with RMSD between predictions generally varying by only a few percent. This might be due to scarcity of apo data in SAbDab, and because very few antibodies are resolved in more than one conformation. It is also possible that the confidence score is not as effective at evaluating unbound protein loops as it is at scoring protein-protein interfaces.

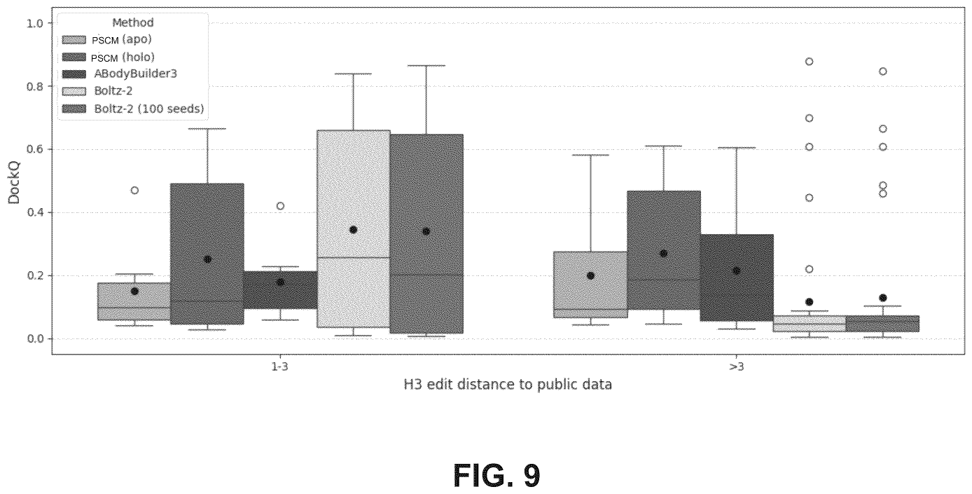

To further evaluate the quality of predicted antibody structures, physics-based docking was performed against their corresponding ground truth antigen structures using HADDOCK344 and using the DockQ score, a metric ranging from 0 (incorrect) to 1 (perfect) that combines interface RMSD, fraction of native contacts, and ligand RMSD. HADDOCK3 was chosen as it is a widely used and validated physics-based docking program that can integrate information, such as the epitope and paratope restraints used here, to guide the docking process. For this analysis, a subset of 44 antibody-antigen complexes was selected from the private dataset. This subset was curated to be non-redundant, with each structure having a unique CDR H3 sequence and a unique antigen, thus providing a diverse test set for docking. In this benchmark, both the predicted antibody and the known antigen were kept rigid to evaluate how well the predicted binding interface, particularly the CDR loops, complements the native antigen epitope. Epitope restraints were extracted from the original complex structure using a 5 Å heavy atom distance cutoff between antigen residues and antibody atoms, while CDR residues served as paratope restraints. The docking workflow consisted of rigid body sampling (20 structures), flexible refinement with reduced sampling factors (5), energy minimization (3 sampling), and clustering analysis. As can be observed in FIG. 9, the protein structure computation model's bound conformation predictions perform best, with an average DockQ score of 0.25, while the ABodyBuilder3 model performs comparatively to unbound conformation predictions, where protein structure computation model is used to predict antibodies in the wrong conformation. Predictions for complexes where the CDR H3 loop has an edit distance of one to three from known SAbDab antibodies (totaling 8 structures) were separated from those with an edit distance of four or more (36 structures). The co-folding methods such as Boltz-2 performed relatively well close to the training data distribution, achieving slightly better average DockQ scores than the protein structure computation model combined with HADDOCK3, but generally do not recover the correct pose on complexes out of distribution, with a high CDR H3 edit distance. The confidence head also provides limited signal in identifying promising predictions from a larger pool.

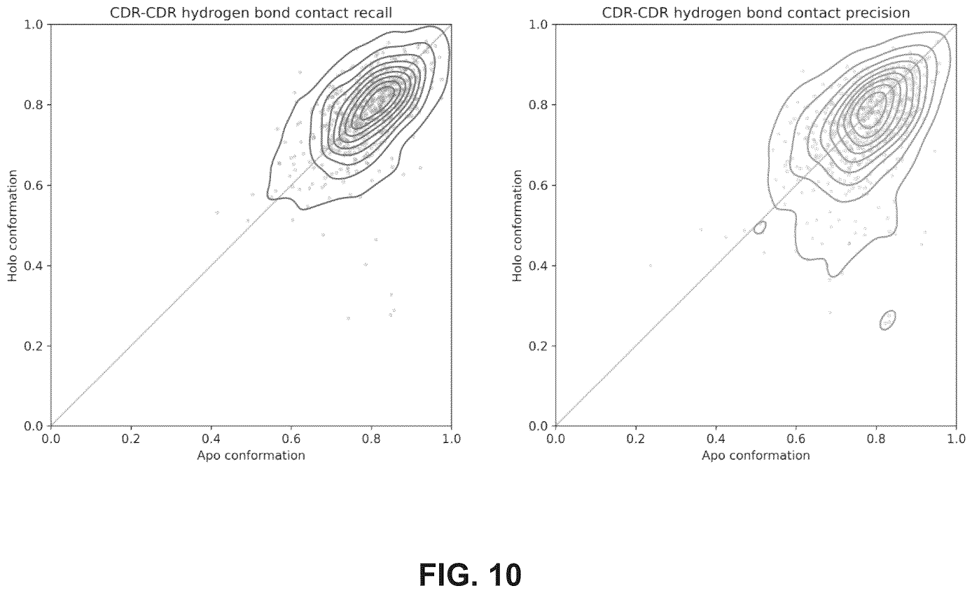

The recall and precision of the protein structure computation model-predicted hydrogen-bond networks within the CDRs was determined relative to the ground truth structures. All coordinates were identically and independently relaxed 5 times using Rosetta to minimize spurious signal between experimental and predicted structures. hydrogen bonds were identified using the Rosetta ScoreFunction as those possessing >10% of the maximum hydrogen-bond energy. For each pair of experimental/predicted structures, a set of unique interacting residues connected by at least one hydrogen bond was computed, sorted by residue order to avoid double counting. The intersection of the two sets yielded true positives (TP) while false positives (FP) and false negatives (FN) were determined from their differences. Precision and recall of the ground truth hydrogen-bond interactions were computed via their standard definitions below. FIG. 10 depicts the recall (A) and precision (B) of hydrogen bond contacts predicted by the protein structure computation model, for pairs of bound/unbound structures.

$$precision = \frac{TP}{TP + FP} , recall = \frac{TP}{TP + FN} . \qquad ( 1 )$$

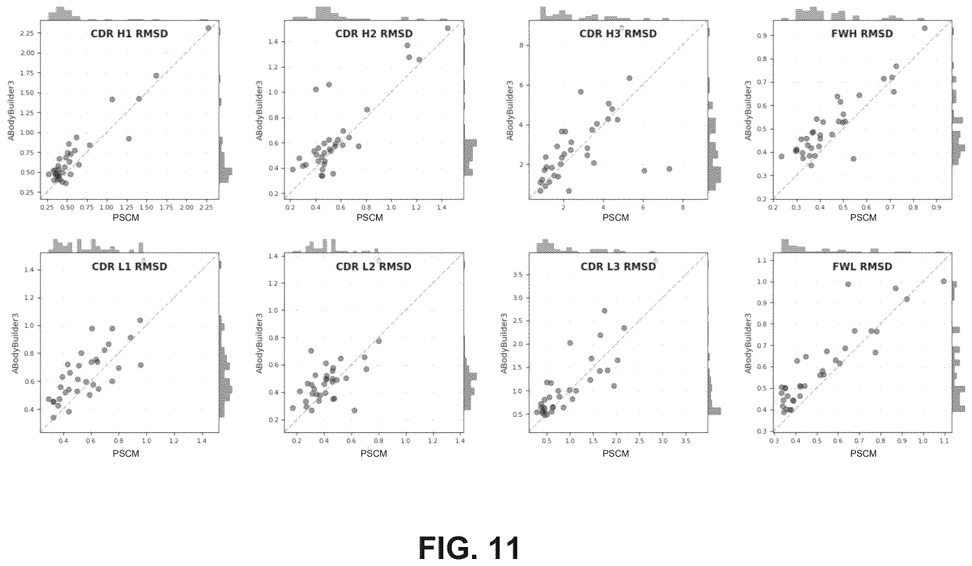

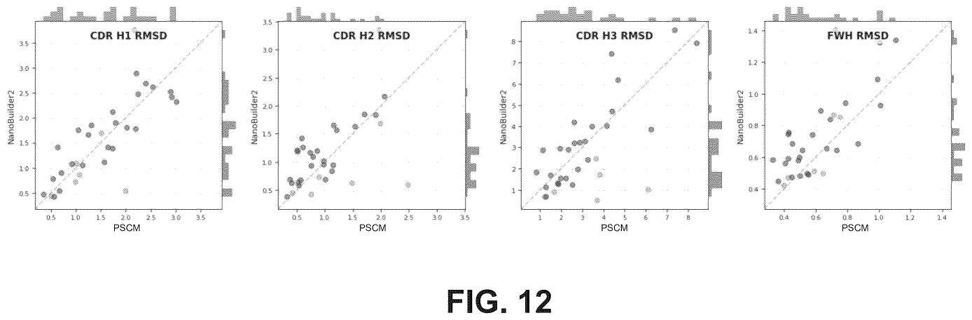

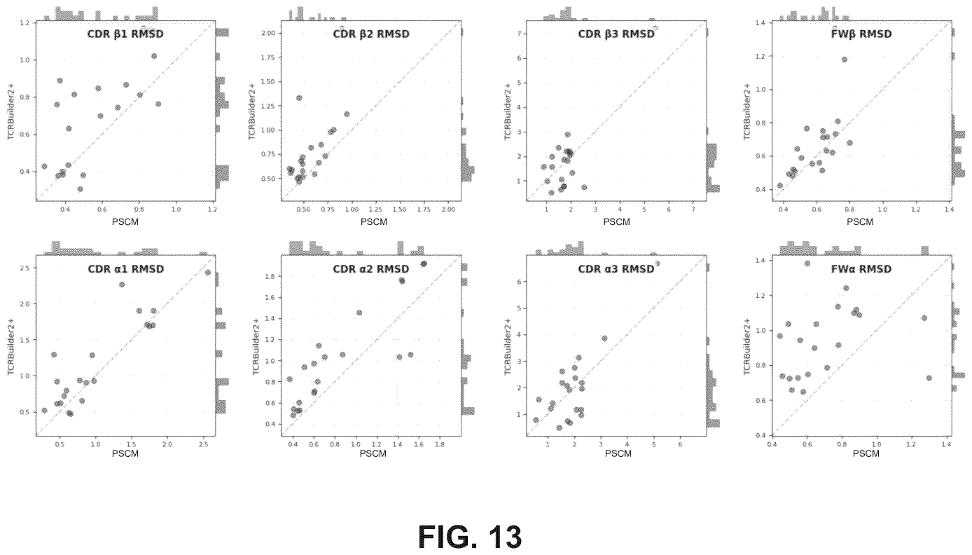

A detailed view of the summary Table 1 is provided here. To compute the RMSD, each predicted heavy and light chain is aligned independently to the corresponding ground truth chain according only to their framework residues. In FIG. 11, the RMSD on the ImmuneBuilder antibody test set is shown separated by region, and comparing the protein structure computation model predictions to ABodyBuilder3. FIG. 12 gives a comparison of the protein structure computation model to NanoBodyBuilder2 on the test set of nanobodies. Here the 6 structures for which an identical match was identified in the train or validation split of NanoBodyBuilder2, which are PDB codes 7n4n, 7omt, 7q6c, 7rg7, 7zmv, and 7zxu, are shown in gray. These points were excluded from the average presented in Table 1. In FIG. 13, a comparison of the Protein structure computation model to TCRBuilder2+ on the test set of 21 TCR structures is shown.

Table 2 provides the average inference time on a typical antibody variable domain for the protein structure computation model, ESMFold, and Boltz-2 on an NVIDIA A10G GPU. Here, Boltz-2 is evaluated with the MSAs having been precomputed and provided as an a3m file, and the compute time used on a node of 8 NVIDIA A100 GPUs to generate these with MMseqs2-GPU is also given. Note that inference time for MSA generation is substantially reduced when batching sequences, and both batched and unbatched timings provided.

| Model | Average Inference Time |
|---|---|
| Protein Structure Computation Model | 0.7 s on single NVIDIA A10G |
| ESMFold | 6.7 s on single NVIDIA A10G |
| Boltz-2, no MSA | 63.9 s on single NVIDIA A10G |
| Boltz-2, with pre-computed MSA | 67.7 s on single NVIDIA A10G |
| MSA, per Fv on a batch of 1000 | 3.5 s on 8 × NVIDIA A100 |
| MSA, single Fv | 10.5 min on 8 × NVIDIA A100 |

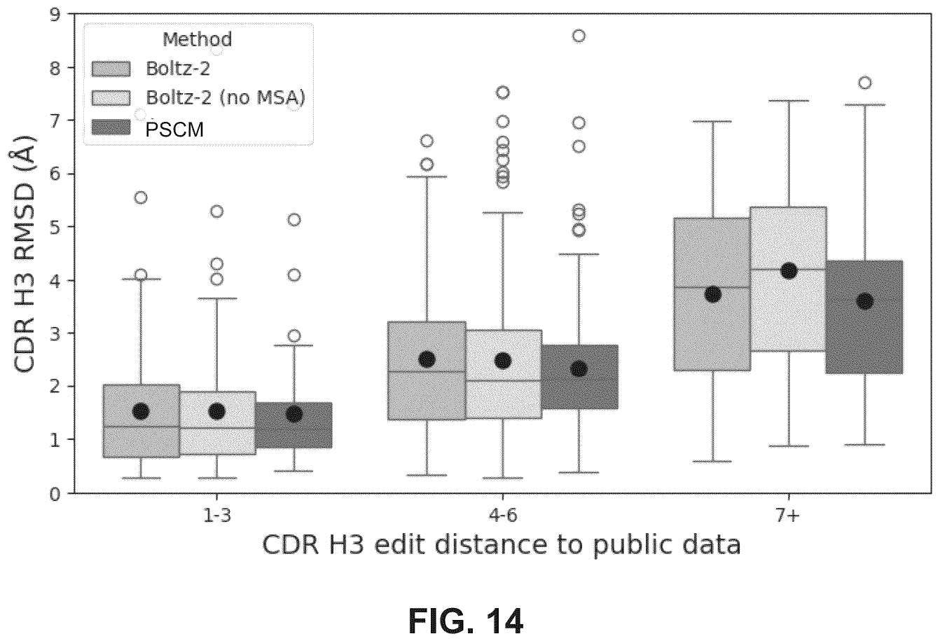

The performance of Boltz-2 is also evaluated on the private dataset when no MSA input is provided to the model. Results are shown in FIG. 14, where it can be observed that while performance is slightly worse out-of-distribution, in the high edit distance bin, providing MSAs leads to no noticeable improvement for antibodies close to known structures.

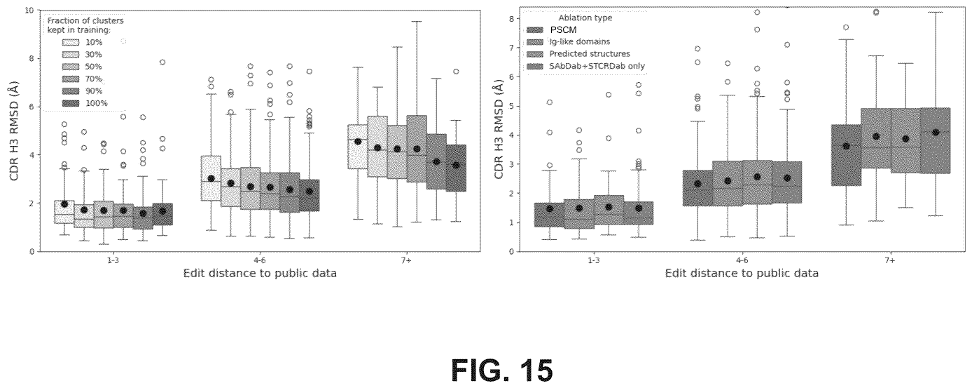

The impact on out-of-distribution robustness of several architecture and dataset choices is considered. To this end, several single model checkpoints were trained for 400 epochs in each three stage. In the left panel of FIG. 15, the impact of dataset size on model performance is depicted. Here, each model is trained on only the experimental antibody and TCR datasets, SAbDab and STCRDab, randomly sub-sampling a fraction of the total available training clusters. Epoch length is rescaled for each model to correspond to the same number of iterations and training time to the model trained on 100% of the dataset. A linear improvement in the high edit distance bin can be observed, suggesting that the collection of further structural data will be beneficial to model robustness and performance. In the right panel of FIG. 15, the results of an ablation studies in which models are trained without the immunoglobulin-like data, without the predicted data, as well as trained only on SAbDab and STCRDab are shown. It can be observed that both the predicted structures and the immunoglobulin-like data lead to improved performance at large edit distance from known public CDR H3 loops, and the full protein structure computation model ensemble model trained on combined data sees additive improvements from both secondary datasets.

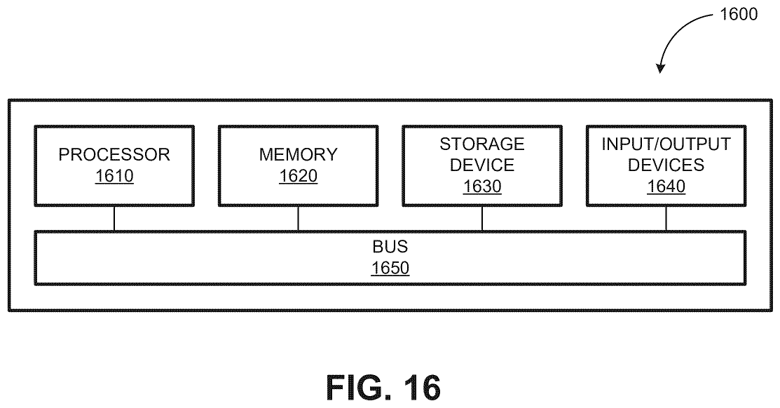

FIG. 16 depicts a block diagram illustrating an example of a computing system 1600, in accordance with some example embodiments. Referring to FIGS. 1-16, the computing system 1600 may be used to implement the structure prediction engine 110, the data store 120, the client device 130, and/or any components therein.

As shown in FIG. 16, the computing system 1600 can include a processor 1610, a memory 1620, a storage device 1630, and input/output devices 1640. The processor 1610, the memory 1620, the storage device 1630, and the input/output devices 1640 can be interconnected via a system bus 1650. The processor 1610 is capable of processing instructions for execution within the computing system 1600. Such executed instructions can implement one or more components of, for example, the structure prediction engine 110, the data store 120, the client device 130, and/or the like. In some example embodiments, the processor 1610 can be a single-threaded processor. Alternately, the processor 1610 can be a multi-threaded processor. The processor 1610 is capable of processing instructions stored in the memory 1620 and/or on the storage device 1630 to display graphical information for a user interface provided via the input/output device 1640.

The memory 1620 is a computer-readable medium such as volatile or non-volatile that stores information within the computing system 1600. The memory 1620 can store data structures representing configuration object databases, for example. The storage device 1630 is capable of providing persistent storage for the computing system 1600. The storage device 1630 can be a floppy disk device, a hard disk device, an optical disk device, or a tape device, or other suitable persistent storage means. The input/output device 1640 provides input/output operations for the computing system 1600. In some example embodiments, the input/output device 1640 includes a keyboard and/or pointing device. In various implementations, the input/output device 1640 includes a display unit for displaying graphical user interfaces.

According to some example embodiments, the input/output device 1640 can provide input/output operations for a network device. For example, the input/output device 1640 can include Ethernet ports or other networking ports to communicate with one or more wired and/or wireless networks (e.g., a local area network (LAN), a wide area network (WAN), the Internet).

In some example embodiments, the computing system 1600 can be used to execute various interactive computer software applications that can be used for organization, analysis and/or storage of data in various formats. Alternatively, the computing system 1600 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 1640. The user interface can be generated and presented to a user by the computing system 1600 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT), a liquid crystal display (LCD), a light emitting diode (LED) monitor, or an organic light emitting diode (OLED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A system, comprising:
at least one data processor; and
at least one memory storing instructions, which when executed by the at least one data processor, result in operations comprising:
receiving an amino acid residue sequence of an input protein molecule;
determining a representation of the amino acid residue sequence of the input protein molecule, wherein the representation is determined to include a conformation token specifying a conformation state of the input protein molecule; and
applying a protein structure computation model to determine, based at least on the representation of the amino acid residue sequence of the input protein molecule, a conformation of the input protein molecule in the conformation state specified by the conformation token.

2. The system of claim 1, wherein the conformation state of the input protein molecule is an unbound state comprising a three-dimensional structure adopted by the input protein molecule absent any interaction with another molecule.

3. The system of claim 1, wherein the conformation state of the input protein molecule is a bound state comprising a three-dimensional structure adopted by the input protein molecule interacting or in complex with another molecule.

4. The system of claim 1, wherein the representation of the amino acid residue sequence of the input protein molecule includes, for each constituent amino acid residue, a residue level encoding of the conformation token.

5. The system of claim 1, wherein the protein structure computation model includes an ensemble of structure module blocks operating on the representation of the amino acid residue sequence of the input protein molecule.

6. The system of claim 5, wherein each structure molecule block in the ensemble of structure module blocks updates, based at least on the representation of the amino acid residue sequence, one or more coordinates of one or more backbone atoms comprising each constituent amino acid residue in the input protein molecule.

7. The system of claim 6, wherein one or more coordinates of one or more sidechain atoms comprising each constituent amino acid residue are determined, using (i) idealized coordinates of a type of each constituent amino acid residue and (ii) one or more torsion angles the one or more sidechain atoms.

8. The system of claim 1, wherein the representation of the amino acid residue sequence of the input protein molecule further includes an embedding of the amino acid residue sequence generated by a protein language model (PLM).

9. The system of claim 1, wherein the representation of the amino acid residue sequence of the input protein molecule includes, for each constituent amino acid residue, a residue-level encoding of an antibody chain in which the amino acid residue is located.

10. The system of claim 1, wherein the representation of the amino acid residue sequence of the input protein molecule includes a positional encoding of a relative position of each constituent amino acid residue.

11. The system of claim 1, wherein the protein structure computation model includes an ensemble of independently trained machine learning models, and wherein each machine learning model in the ensemble of machine learning models is applied to determine the conformation of the input protein molecule.

12. The system of claim 11, wherein the operations further comprise:
aligning a plurality of outputs from the ensemble of machine learning models;
determining a mean conformation based on the aligned plurality of outputs from the ensemble of machine learning models; and
determining the conformation of the input protein molecule to correspond to an output from a machine learning model in the ensemble of machine learning models that is closest to the mean conformation.

13. The system of claim 1, wherein the protein structure computation model includes a residual connection to every structure module block comprising the protein structure computation model to preserve the conformation token while the protein structure computation model operates on the representation of the input protein molecule.

14. The system of claim 1, wherein the operations further comprise:
generating a training sample to include an amino acid residue sequence of a sample protein molecule and a ground truth conformation of the sample protein molecule in an unbound state;
generating an additional training sample to include the amino acid residue sequence of the sample protein molecule and a ground truth conformation of the sample protein molecule in a bound state;
generating a training dataset to include the training sample and the additional training sample; and
training the protein structure computation model on the training dataset.

15. The system of claim 14, wherein one or more of the ground truth conformation of the sample protein molecule in the unbound state and the ground truth conformation of the sample protein molecule in the bound state comprise a crystallized protein structure.

16. The system of claim 14, wherein one or more of the ground truth conformation of the sample protein molecule in the unbound state and the ground truth conformation of the sample protein molecule in the bound state comprise a computationally generated protein structure.

17. The system of claim 1, wherein the operations further comprise:
applying a protein design computation model to generate the amino acid residue sequence of the input protein molecule, wherein the protein design computation model generates the amino acid residue sequence of the input protein molecule by modifying (i) an amino acid residue sequence of a lead molecule having one or more known properties or (ii) a noise sequence without any known properties.

18. The system of claim 1, wherein the input protein molecule comprises one or more of a complementarity determining region (CDR), a framework region, a variable region (or fragment antigen binding (Fab)), a constant region (or fragment crystallizable (Fc)), a light chain, or a heavy chain.

19. A computer-implemented method, comprising:
receiving an amino acid residue sequence of an input protein molecule;
determining a representation of the amino acid residue sequence of the input protein molecule, wherein the representation is determined to include a conformation token specifying a conformation state of the input protein molecule; and
applying a protein structure computation model to determine, based at least on the representation of the amino acid residue sequence of the input protein molecule, a conformation of the input protein molecule in the conformation state specified by the conformation token.

20. A non-transitory computer readable medium storing instructions, which when executed by at least one data processor, result in operations comprising:
receiving an amino acid residue sequence of an input protein molecule;
determining a representation of the amino acid residue sequence of the input protein molecule, wherein the representation is determined to include a conformation token specifying a conformation state of the input protein molecule; and
applying a protein structure computation model to determine, based at least on the representation of the amino acid residue sequence of the input protein molecule, a conformation of the input protein molecule in the conformation state specified by the conformation token.
